(19) United States
(12) Patent Application Publication
(10) Pub. No.: US 2023/0263448 A1
(43) Pub. Date: Aug. 24, 2023

(54) SIX LEAD ECG DEVICE WITH A REDUCED FORM FACTOR

(71) Applicant: AliveCor, Inc., Mountain View, CA (US)

(72) Inventors: Sean Cohen, Mountain View, CA (US); Siva Somayajula, Saratoga, CA (US); David E. Albert, Oklahoma City, OK (US)

(21) Appl. No.: 18/161,641

(22) Filed: Jan. 30, 2023

Related U.S. Application Data
(60) Provisional application No. 63/304,511, filed on Jan. 28, 2022.

(51) Int. Cl. A61B 5/282; A61B 5/339; A61B 5/327; A61B 5/00; A61B 5/346; A61B 90/96

(52) U.S. Cl. CPC A61B 5/282; A61B 5/339; A61B 5/327; A61B 5/0006; A61B 5/346; A61B 90/96

(57) ABSTRACT

Described herein is an apparatus for monitoring various physiological parameters of a user. The apparatus may include a housing with a small or reduced form factor near the size of a credit or debit card, so as to allow a user to obtain e.g., an electrocardiogram (ECG) measurement from various locations. The apparatus may include an electrode assembly comprising a set of electrodes disposed on the housing to perform an ECG by sensing an electrical signal corresponding to heart activity of a user when in contact with skin of the user and output the electrical signal. The apparatus may detect or generate six or more ECG leads.

### CROSS-REFERENCE

This application claims the benefit of U.S. Provisional Application No. 63/304,511, filed Jan. 28, 2022, and entitled “SIX LEAD ECG DEVICE WITH A REDUCED FORM FACTOR,” the disclosure of which is hereby incorporated by reference.

### TECHNICAL FIELD

The present disclosure relates to medical devices, systems, and methods and in particular, to reduced form factor devices for providing electrocardiogram (ECG) monitoring.

### BACKGROUND

Cardiovascular diseases are the leading cause of death in the world. In 2008, 30% of all global death were attributed to cardiovascular diseases. It is also estimated that by 2030, over 23 million people will die from cardiovascular diseases annually. Cardiovascular diseases are prevalent across populations of first and third world countries alike, and affect people regardless of socioeconomic status.

Arrhythmia is a cardiac condition in which the electrical activity of the heart is irregular or is faster (tachycardia) or slower (bradycardia) than normal. Although many arrhythmias are not life-threatening, some can cause cardiac arrest and even sudden cardiac death. Indeed, cardiac arrhythmias are one of the most common causes of death when travelling to a hospital. Atrial fibrillation (A-fib) is the most common cardiac arrhythmia. In A-fib, electrical conduction through the ventricles of heart is irregular and disorganized. While A-fib may cause no symptoms, it is often associated with palpitations, shortness of breath, fainting, chest pain or congestive heart failure and also increases the risk of stroke. A-fib is usually diagnosed by taking an electrocardiogram (ECG) of a subject. To treat A-fib, a patient may take medications to slow heart rate or modify the rhythm of the heart. Patients may also take anticoagulants to prevent stroke or may even undergo surgical intervention including cardiac ablation to treat A-fib. In another example, an ECG may provide decision support for Acute Coronary Syndromes (ACS) by interpreting various rhythm and morphology conditions, including Myocardial Infarction (MI) and Ischemia.

Often, a patient with A-fib (or other type of arrhythmia) is monitored for extended periods of time to manage the disease. For example, a patient may be provided with a Holter monitor or other ambulatory electrocardiography device to continuously monitor the electrical activity of the cardiovascular system for e.g., at least 24 hours. Such monitoring can be critical in detecting conditions such as acute coronary syndrome (ACS), among others.

Prehospital ECG has been found to significantly reduce time-to-treatment for patients with possible ACS when symptoms present and shows better survival rates. The time-to-first-ECG is so vital that it is a quality and performance metric monitored by several regulatory bodies. According to the national health statistics for 2015, over 7 million people visited the emergency department (ED) in the United States (U.S.) with the primary complaint of chest pain or related symptoms of ACS. In the U.S., ED visits are increasing at a rate of or 3.2% annually and outside the U.S. ED visits are increasing at 3% to 7%, annually.

### BRIEF DESCRIPTION OF THE DRAWINGS

The present invention is illustrated by way of example and not limitation in the figures of the accompanying drawings in which like references indicate similar elements.

FIG. 1 illustrates a 3 lead ECG waveform as well as an exploded view of the ECG waveform measured by one of the leads III that illustrates a QRS complex, according to an embodiment of the present disclosure.

FIG. 2 illustrates a single dipole heart model with a 12-lead set represented on a hexaxial system, according to an embodiment of the present disclosure.

FIG. 3 illustrates a system in which embodiments of the present disclosure may be realized.

FIG. 4A shows a perspective view of an example form factor of a housing of a monitoring device for remote or mobile acquisition of ECG data, according to an embodiment of the present disclosure.

FIG. 4B illustrates a topside of a lower layer of the housing of FIG. 4A, according to an embodiment of the present disclosure.

FIG. 4C illustrates a topside of an upper layer of the housing of FIG. 4A, according to an embodiment of the present disclosure.

FIG. 4D illustrates a bottom side of a lower layer of the housing of FIG. 4A, according to an embodiment of the present disclosure.

FIG. 4E illustrates a topside of an upper layer and a bottom side of a lower layer of the housing of FIG. 4A, with the top layer including a visual/machine-readable representation of data, according to an embodiment of the present disclosure.

FIG. 4F shows a flow diagram of an example method of obtaining and providing remote ECG data using an ECG device with a portable form factor, according to an embodiment of the present disclosure.

FIG. 5 illustrates an example computer system which may be used in conjunction with the embodiments described herein.

### DETAILED DESCRIPTION

It is to be understood that the present disclosure is not limited in its application to the details of construction, experiments, exemplary data, and/or the arrangement of the components set forth in the following description. The embodiments of the present disclosure are capable of other embodiments or of being practiced or carried out in various ways. Also, it is to be understood that the terminology employed herein is for purpose of description and should not be regarded as limiting.

In the following detailed description of embodiments of the present disclosure, numerous specific details are set forth in order to provide a more thorough understanding of the disclosure. However, it will be apparent to one of ordinary skill in the art that the concepts within the disclosure can be practiced without these specific details. In other instances, well-known features have not been described in detail to avoid unnecessarily complicating the description.

An electrocardiogram (ECG) provides a number of ECG waveforms that represent the electrical activity of a person's heart. An ECG monitoring device may comprise a set of electrodes for recording the ECG waveforms (also referred to herein as “taking an ECG”) of the patient's heart. The skin of the patient may come into contact with the set of electrodes at multiple locations, and the electrical signal recorded between each electrode pair in the set of electrodes may be referred to as a lead. Varying numbers of leads can be used to take an ECG, and different numbers and combinations of electrodes can be used to form the various leads. Example numbers of leads used for taking ECGs are 1, 2, 6, and 12 leads.

The ECG waveforms (each one corresponding to a lead of the ECG) recorded by the ECG monitoring device may comprise data corresponding to the electrical activity of the person's heart. A typical heartbeat may include several variations of electrical potential, which may be classified into waves and complexes, including a P wave, a QRS complex, a T wave, and a U wave among others, as is known in the art. Stated differently, each ECG waveform may include a P wave, a QRS complex, a T wave, and a U wave among others, as is known in the art. The shape and duration of these waves may be related to various characteristics of the person's heart such as the size of the person's atrium (e.g., indicating atrial enlargement) and can be a first source of heartbeat characteristics unique to a person. The ECG waveforms may be analyzed (typically after standard filtering and “cleaning” of the signals) for various indicators that are useful in detecting cardiac events or status, such as cardiac arrhythmia detection and characterization. Such indicators may include ECG waveform amplitude and morphology (e.g., QRS complex amplitude and morphology), R wave-ST segment and T wave amplitude analysis, and heart rate variability (HRV), for example.

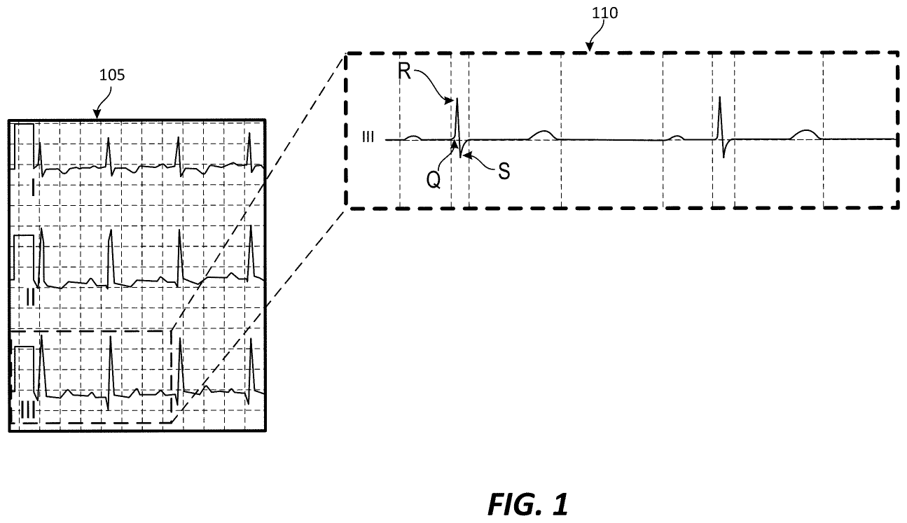

As noted above, ECG waveforms are generated from measuring multiple leads (each lead formed by a different electrode pair), and the ECG waveform obtained from each different electrode pair/lead may be different/unique (e.g., may have different morphologies/amplitudes). This is because although the various leads may analyze the same electrical events, each one may do so from a different angle. FIG. 1 illustrates a view 105 of an ECG waveform detected by each of 3 leads (I, II, and III) when a 3-lead ECG is taken as well as an exploded view 110 of the ECG waveform measured by lead III illustrating the QRS complex. As shown, the amplitudes and morphologies of the ECG waveform taken from leads I-III are all different, with the ECG waveform measured by lead III having the largest amplitude and the ECG waveform measured by lead I having the smallest amplitude.

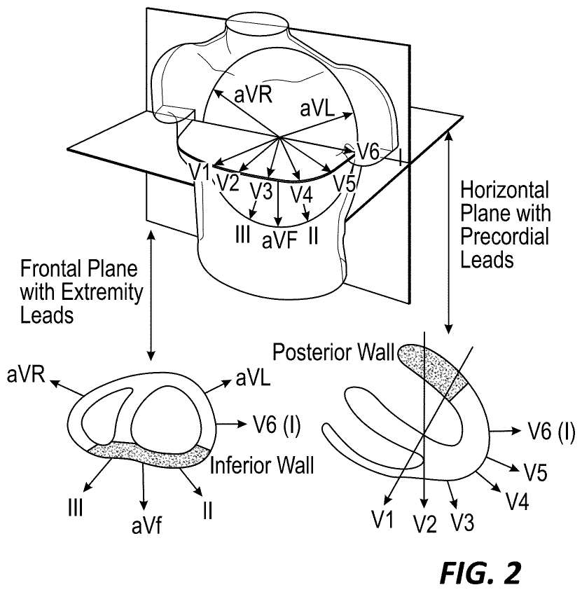

FIG. 2 illustrates a single dipole heart model 115 with a 12 lead set comprising the I, II, III, aVR, aVL, aVF, V1, V2, V3, V4, V5, and V6 leads, all represented on a hexaxial system. The heart model 115 assumes a homogeneous cardiac field in all directions that only changes magnitude and direction with the cycle time. As illustrated in FIG. 1B, there are 2 orthogonal planes, the frontal plane and the horizontal plane. Inside each plane, there are several leads to cover the whole plane. In the frontal plane, there are 2 independent leads I and II, and 4 other derived leads III, aVR, aVL, and aVF, each 30 degrees apart. The reason the frontal plane has 2 independent leads is that they are far-field leads, each of which can cover a wider perspective but provide less detail, like a wide-angle camera lens. In the horizontal plane, there are normally 6 independent leads which are all closer to the heart than limb leads and may be referred to as near-field leads. Following the same analogy of a camera, the near-field leads may behave like a zoom-lens that covers less perspective, but with more accuracy towards local activity like ischemia and infarction. The two orthogonal planes are related by using a synthetic reference point formed by Leads I & II, called the Wilson-Central-Terminal (WCT). It is defined as RA+LA+RL/3 but given that both Lead I and II are recorded with reference to the RA so that the voltage of the RA can be considered zero, the WCT (VW) can be calculated using the RA as the reference for both Leads I & II (thus, assuming it to have zero potential) as:

Lead I+Lead II/3.

There are different “standard” configurations for where electrodes may contact the patient. For example, an electrode in contact with the right arm can be referred to as RA. The electrode in contact with the left arm can be referred to as LA. The RA and LA electrodes may be in contact with the same location on the left and right arms, preferably near the wrist in some embodiments. The leg electrodes can be referred to as RL for the right leg and LL for the left leg. The RL and LL electrodes may be in contact with the same location for the left and right legs, preferably near the ankle in some embodiments. Lead I is typically the voltage between the left arm (LA) and right arm (RA), e.g. I=LA−RA. Lead II is typically the voltage between the left leg (LL) and right arm (RA), e.g. II=LL−RA. Lead III is the typically voltage between the left leg (LL) and left arm (LA), e.g. III=LL−LA. Augmented limb leads can also be determined from RA, RL, LL, and LA. The augmented vector right (aVR) lead is equal to RA−(LA+LL)/2 or −(I+II)/2. The augmented vector left (aVL) lead is equal to LA−(RA+LL)/2 or I−II/2. The augmented vector foot (aVF) lead is equal to LL−(RA+LA)/2 or II−I/2. Thus, a 6-lead ECG may be obtained under a standard electrode configuration by using three or more electrodes to measure three voltage differences (leads I, II, and II) and deriving three augmented vectors (aVR, aVL, and aVF).

As discussed herein, a 6-lead ECG may be acquired as early as possible for patients with possible ACS when symptoms present because prehospital ECG has been found to significantly reduce time-to-treatment and shows better survival rates. In addition, current ambulatory ECG devices such as Holter monitors, are typically bulky and difficult for subjects to administer without the aid of a medical professional. For example, the use of a Holter monitor requires a patient to wear a bulky device on their chest and precisely place a plurality of electrode leads on precise locations on their chest. These requirements can impede the activities of the subject, including their natural movement such as bathing and showering. Once an ECG is taken by such devices, the ECG is sent to the subject's physician who then analyzes the ECG waveforms and provides a diagnosis and other recommendations. Currently, this process is often performed through hospital administrators and health management organizations and many patients do not receive feedback in an expedient manner.

A number of handheld ECG measurement devices are known, including devices that may adapt existing mobile telecommunications devices (e.g., smartphones) so that they can be used to record an ECG. However, such devices either require the use of external (e.g., plug-in) electrodes, or include electrodes in a housing that are difficult to properly hold and apply to the body. Many ECG monitors are also limited to acquiring limb leads (e.g., due to size and other constraints. Obtaining a V lead not only provides an additional channel of ECG measurement, it has potential to add another orthogonal cardiac field plane, called the horizontal plane, thanks to the reference point formed by leads I and II. This is important because as people age, their QRS and T-wave vector may gradually move from the frontal plane to the horizontal plane, thus increasing the importance of acquiring data from a horizontal plane lead.

Embodiments of the present disclosure address the above and other problems by providing a 6-lead ECG monitoring device (hereinafter referred to as an ECG monitoring device) having a credit card-like form factor that allows it to be carried on a person (e.g., in their wallet or purse) as readily as a standard credit card. The ECG monitoring device described herein may acquire 3 standard ECG leads and derive three augmented leads, while not requiring the use of adhesives for electrodes. The ECG monitoring device can be used by a user/patient, and provides ECG data to a user on a near instantaneous basis. For example, the ECG monitoring device may acquire leads I, II, and III and derive leads aVR, aVL, and aVF. However, any other combination of leads is possible. The ECG monitoring device may subsequently generate a 12-lead ECG using the three measured leads.

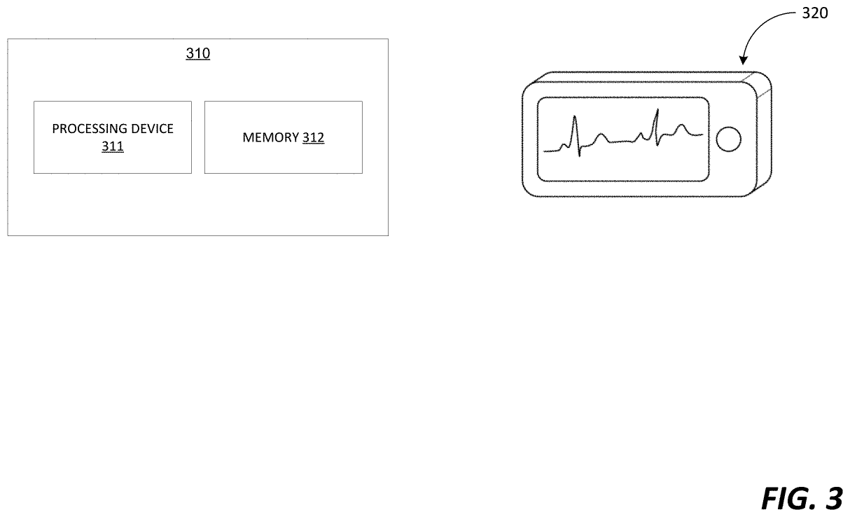

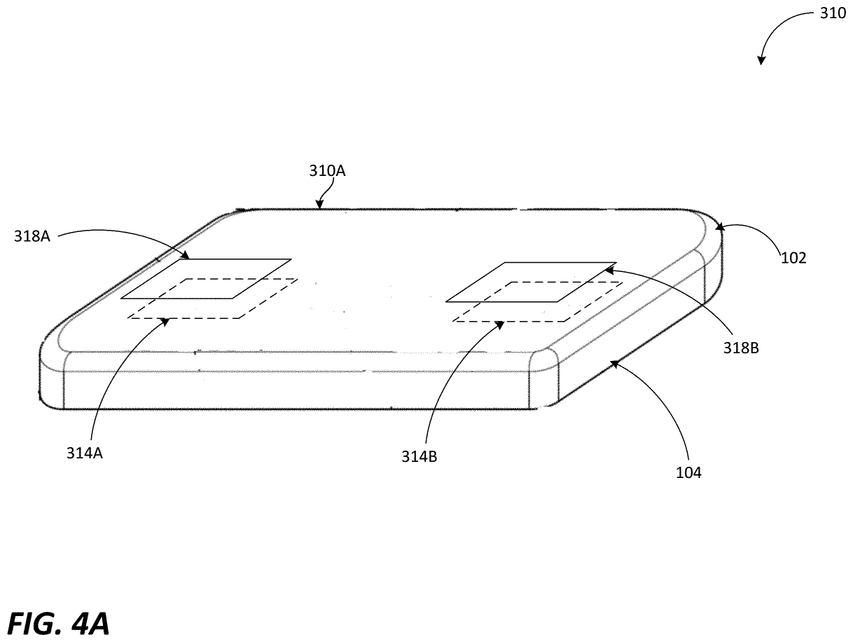

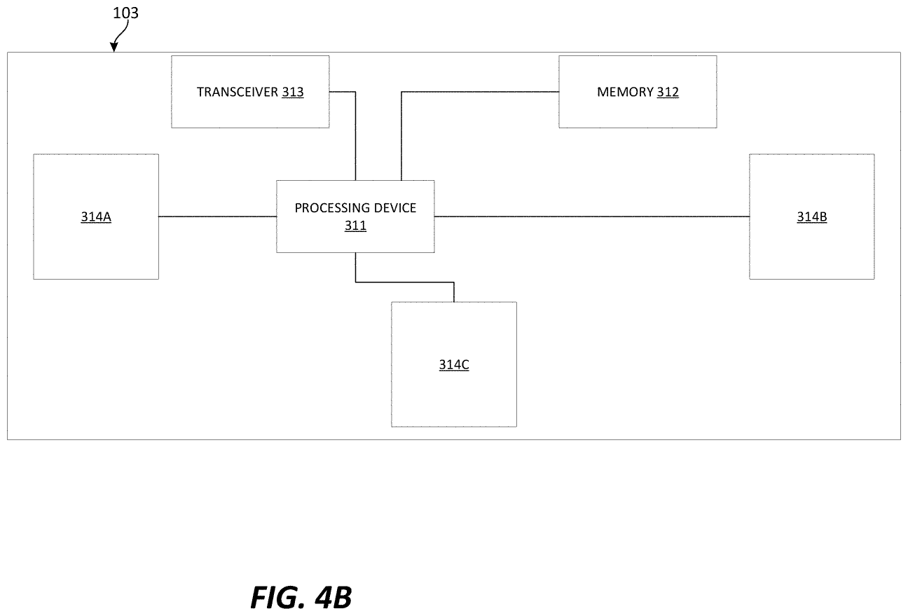

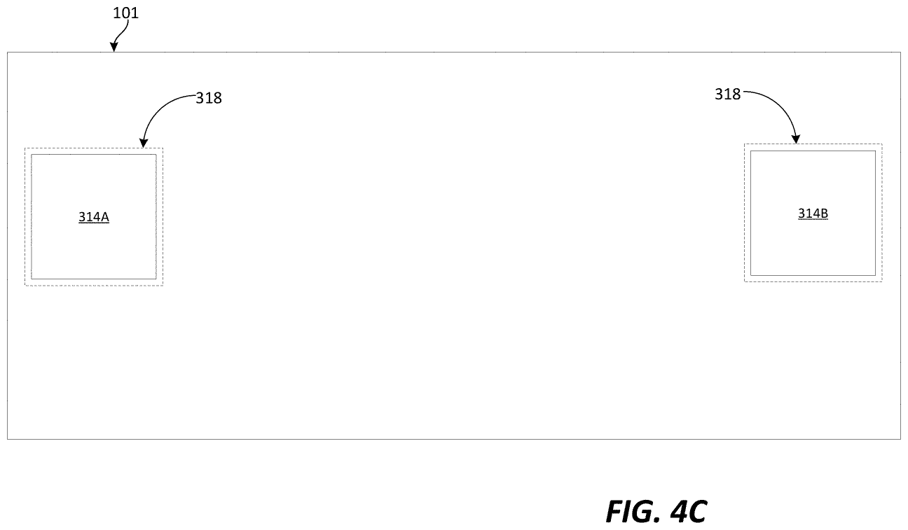

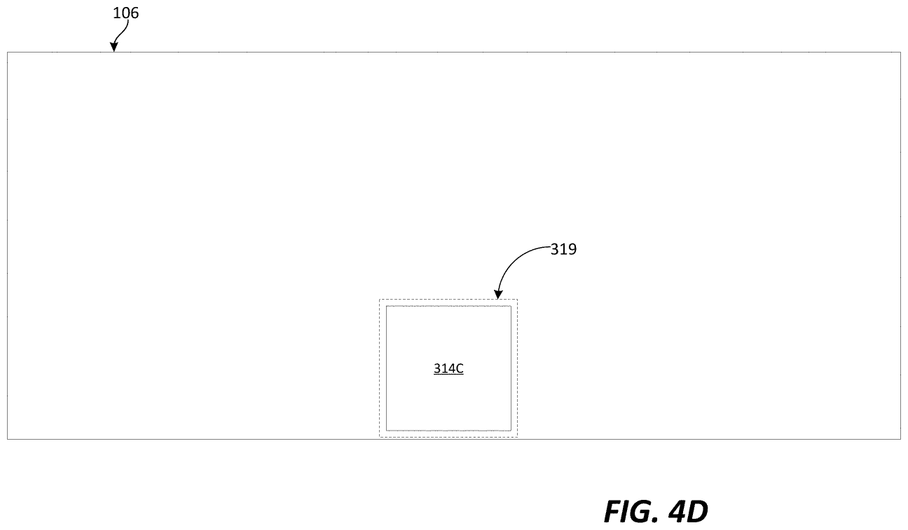

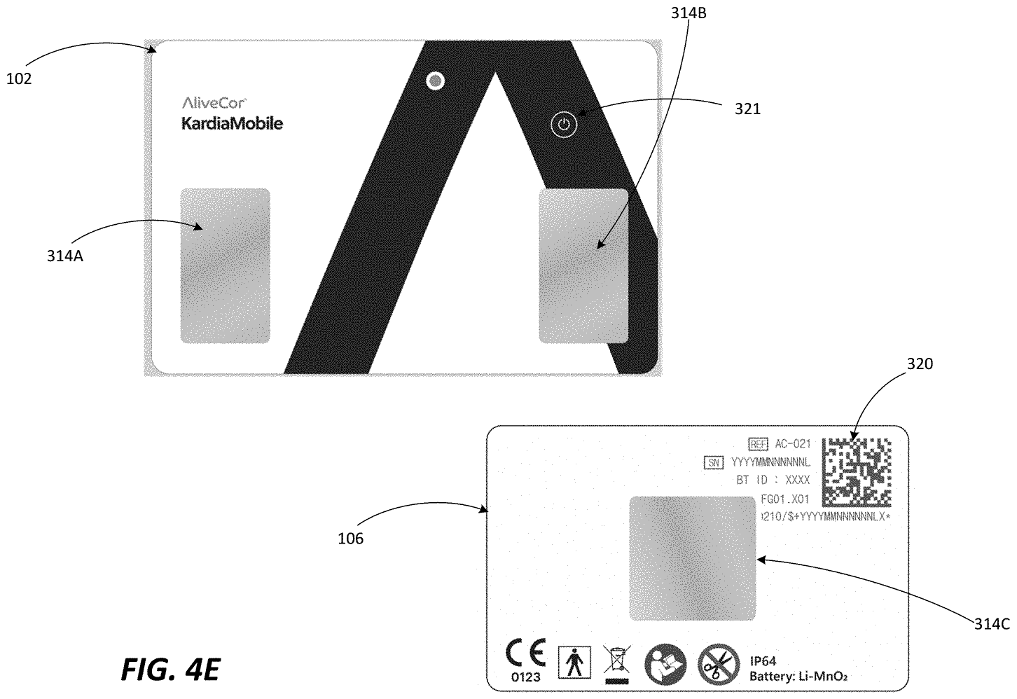

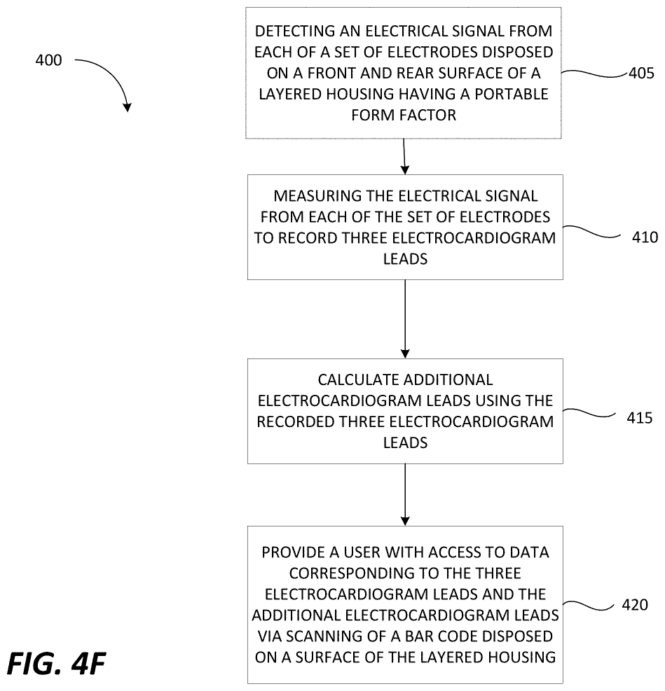

FIG. 3 illustrates a system 300 in which embodiments of the present disclosure may be realized. System 300 may include a monitoring device 310 as well as a computing device 320. Monitoring device 310 may include a set of electrodes (shown in FIGS. 4A-4E) for measuring multiple ECG leads as discussed in further detail herein. Each of the monitoring device 310 and computing device 320 may comprise hardware for performing the respective functions described herein such as processing device 311 (e.g., processors, central processing units (CPUs)), memory 312 (e.g., random access memory (RAM), storage devices (e.g., hard-disk drive (HDD)), solid-state drives (SSD), etc.), and other hardware devices (e.g., analog to digital converter (ADC) etc.). A storage device may comprise a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. In some embodiments, the processing device 311 may comprise a dedicated ECG waveform processing and analysis chip that provides built-in leads off detection. The monitoring device 310 may include an ADC (not shown) having a high enough sampling frequency for accurately converting the ECG waveforms measured by the set of electrodes into digital signals (e.g., a 24 bit ADC operating at 500 Hz or higher) for processing by the processing device 311. In some embodiments, the ADC may be implemented as part of the processing device 311.

The monitoring device 310 may further comprise a transceiver 313, which may implement any appropriate protocol for transmitting ECG data wirelessly to one or more local and/or remote computing devices such as computing device 320. For example, the transceiver 313 may comprise a Bluetooth™ chip for transmitting ECG data via Bluetooth to computing device 320 or other local computing devices (e.g., a laptop or smart phone of the user). In other embodiments, the transceiver 313 may include (or be coupled to) a network interface device configured to connect with a cellular data network (e.g., using GSM, GSM plus EDGE, CDMA, quadband, or other cellular protocols) or a WiFi (e.g., an 802.11 protocol) network, in order to transmit the ECG data to computing device 320 or another remote computing device (e.g., a computing device of a physician or healthcare provider) and/or a local computing device. The monitoring device 310 may comprise a housing 310A within which all of the components of the monitoring device 310 (discussed in further detail herein) may be housed.

FIG. 4A shows a perspective view of an example form factor of a housing 310A of the monitoring device 310 for remote or mobile acquisition of ECG data. As shown in FIG. 4A, the housing 310A may have a form factor similar to that of a credit or debit card. The housing 310A may be flexible and made of any appropriate material. In some embodiments, the monitoring device 310 may be made of a plastic or polymer, such as polyvinyl chloride acetate (PVCA). The form factor of the housing 310A may have a thickness between e.g., 0.65 mm to 0.85 mm (e.g., approximately 0.75 mm), or any other appropriate thickness. Some embodiments of the monitoring device 310 may have a thickness between 0.65 mm to 0.85 mm, while other embodiments may have a thickness between 0.70-0.78 mm. The housing 310A may also have a range of bending stiffness, and may meet the standards outlined by the ISO 7810 ID-1 format. In some embodiments, the housing 310A may have a level of bending stiffness or flexibility that permits a user to place it in a purse or wallet in a similar manner to how a normal credit or debit card is stored and carried.

As shown in FIG. 4A, the housing 310A may have a sandwich structure with an upper layer 102 and a lower layer 104. FIG. 4B illustrates a topside 103 of the lower layer 104. The topside 103 may comprise (e.g., have embedded on it) electrodes 314A-314C, with the electrodes 314A and 314B each located on a respective side of the housing 310A. The electrode 314C may be positioned in the center of the housing 310A, either in proximity to the bottom of the housing 310A (as shown in FIG. 4B) or in proximity to the top of the housing 310A. Each of the electrodes 314 may be any appropriate type of electrode, such as e.g., flexible membrane electrodes or conductive metal (e.g., stainless steel) electrodes. The topside 103 may also comprise (e.g., have embedded on it) processing device 311 (which may be coupled to each of the electrodes 314), memory 312, transceiver 313, and a battery 315, which are all coupled to the processing device 311. The topside 103 may further include (e.g., have mounted thereon) features such as a power button 321 and an LED indicator 322 (both shown in FIG. 4E). The battery 315 may power the other components of the monitoring device 310.

In some embodiments, the topside 103 may also comprise flexible membrane pads (not shown in the FIGS.). Each flexible membrane pad may be positioned under a corresponding electrode 314. In some embodiments, each flexible membrane pad may be positioned directly under a corresponding electrode 314, while in other embodiments, the position of each flexible membrane pad may be offset from the position of the corresponding electrode 314. Each flexible membrane pad may be electrically connected to a corresponding electrode 314 and may receive the electrical signals sensed by the corresponding electrode 314. Each flexible membrane pad may also be electrically coupled to the processing device 311 and may transmit the electrical signals sensed by the corresponding electrode 314 to the processing device 311.

FIG. 4C depicts a topside 101 of upper layer 102. The topside 101 may expose may have openings to expose) the electrodes 314A and 314B and the power button 321 so that they can be contacted by a user, as well as the LED indicator 322. In some embodiments, the topside 101 may comprise touch pads 318 that are each in electrical contact with a respective electrode 314. In such embodiments, the user may contact the touch pads 318 (e.g., with their left and right fingers) in order to perform an ECG.

FIG. 4D illustrates a bottom side 106 of the lower layer 104. As can be seen, the bottom side 106 may expose (e.g., may have an opening to expose) the electrode 314C so that it can be contacted by a user. In some embodiments, the electrode 314C may protrude beyond the edge of the bottom side 106 for better contact with the skin of the user. In some embodiments, the bottom side 106 may comprise a touch pad 319 that is in electrical contact with the electrode 314C. In the example of FIGS. 4A-4D, the electrode 314A may be a left arm (LA) electrode, the electrode 314B may be a right arm (RA) electrode, and the electrode 314C may be a left leg (LL) electrode, or any appropriate chest (V lead) electrode so as to take a non-standard configuration ECG.

Although the components of the monitoring device 310 are described and illustrated as being mounted on the topside 103 of the lower layer 104, this is an example only and the components of monitoring device 310 may be mounted on a bottom side of the upper layer 102. In some embodiments, the electrodes 314A and 314B may be mounted on the topside 101 while the electrode 314C may be mounted on the bottom side 106. For example, the electrodes 314A-C may be made of conductive metal (e.g., stainless steel) that is attached to the topside 101 (i.e., the exterior surface of the upper layer 102 for electrodes 314A and 314B) and the bottom side 106 for electrode 314C. In other examples, the electrodes 314A-C may be fabricated using conductive ink, which is then deposited onto the topside 101 and bottom side 106. The conductive ink may be deposited onto the topside 101 and bottom side 106 in such a fashion that the user is not aware of the presence of the electrodes 314A and 314B during operation of the monitoring device 310. In addition, in some embodiments the monitoring device 310 may have only a single layer on which all of the components of monitoring device 310 are mounted.

In some embodiments, instead of being mounted directly to the housing 310A, the electrodes 314, processing device 311, memory 312, transceiver 313, and the battery 315 may all be integrated into an inlay (not shown). The inlay may comprise a substrate, or sheet carrier, to which the electrodes 314, processing device 311, memory 312, transceiver 313, and the battery 315 are all integrated. The substrate may be any appropriate plastic or polymer material, such as polyethylene terephthalate (PET). In some embodiments, one side of the inlay may be coated with an adhesive which may allow it to be bonded to the top side 103. In other embodiments, the inlay may be integrated to the top side 103 using a process called lamination. Although illustrated as separate components, one or more of the processing device 311, memory 312, battery 315, power button 321, and transceiver 313 may be integrated into a single unit, such as a microcontroller.

FIG. 4E illustrates renderings of the topside 101 of the upper layer 102 and the bottom side 106 of the lower layer 104. As shown in FIG. 4E, in some embodiments the bottom side 106 may include a bar code 320 which may comprise a one-dimensional bar code, two-dimensional bar code (e.g., a QR code) or any other similar type of visual/machine-readable representation of data. The bar code 320 may be scanned by any appropriate device (e.g., smart phone) to retrieve ECG information about the user of the monitoring device 310. The bar code 320 may be linked to a database (e.g., on the computing device 320 or another computing device(s) representing a cloud storage service) Where ECG information about the user may be stored.

The monitoring device 310 may be operated using a standard configuration where the monitoring device 310 may take a 3-lead ECG (leads I, II, and III) using the processing device 311. To take an ECG, a user may hold the monitoring device 310 such that the user's right hand (or e.g., a finger thereof) contacts electrode 314B (RA electrode) while the user's left hand (or e.g., a finger thereof) contacts electrode 314A (LA electrode). In addition, the user may hold the monitoring device 310 in such a way that electrode 314C (LL electrode) contacts the user's left leg.

The monitoring device 310 may take the 3-lead ECG in a standard configuration of the user (by utilizing the processing device 311 to measure the signal generated by the electrodes 314 and simultaneously record leads I, II, and III). The processing device 311 may perform analog to digital conversion and perform any additional signal processing/cleaning functions necessary before utilizing the transceiver 313 to transmit the measured/digitized/processed signals to the computing device 320. The computing device 320 may subsequently derive any number of additional leads as discussed hereinabove. For example, the computing device 320 may synthesize the aVR, aVL, and aVF leads and then the V1, V2, V3, V4, V5, and V6 leads based on the I, II, III, aVR, aVL, and aVF leads using a lead conversion ML model (e.g., a state space model transform or neural network) to reconstruct a standard 12-lead ECG. In some embodiments, the processing device 311 may include firmware/logic (or the memory 312 may include an appropriate software module which may be executed by the processing device 311) to perform the above-described lead derivations (e.g., automatically derive the aVR, aVL, and aVF leads upon recording leads I, II, and III and synthesize any appropriate number of the V leads).

The monitoring device 310 may also take the three-lead ECG in a non-standard configuration of the user by utilizing the processing device 311 to measure the signal generated by the electrodes 314 and simultaneously record leads I, II, and V2. To take the ECG in a non-standard configuration of the user, the user may hold the monitoring device 310 such that the user's right hand (or e.g., a finger thereof) contacts electrode 314B (RA electrode) while the user's left hand (or e.g., a finger thereof) contacts electrode 314A (LA electrode). In addition, the user may hold the monitoring device 310 in such a way that electrode 314C contacts the V2 position of the user's chest. However, the monitoring device 310 may be placed in other locations on the user's chest to facilitate recording a different V lead as opposed to recording lead V2. The processing device 311 may transmit the recorded signals to the computing device 320 which may subsequently derive leads III, aVR, aVL, and aVF as discussed hereinabove. More specifically, the processing device 311 may perform analog to digital conversion as well as any additional signal processing/cleaning functions necessary before utilizing the transceiver 313 to transmit the measured/digitized/processed signals to the computing device 320. The computing device 320 may subsequently derive any number of additional leads as discussed hereinabove. For example, the computing device 320 may synthesize the, aVR, aVL, and aVF leads and then the V1, V3, V4, V5, and V6 leads based on the V2, I, II, III, aVR, aVL, and aVF leads using a lead conversion ML model (e.g., a state space model transform or neural network) to reconstruct a standard 12-lead ECG. In some embodiments, the processing device 311 may include firmware/logic (or the memory 312 may include an appropriate software module which may be executed by the processing device 311) to derive the III, aVR, aVL, and aVF leads upon recording leads I, II, and V2 and may optionally synthesize any appropriate number of the remaining V leads.

In some embodiments, the monitoring device 310 may have a display (not shown) that allows near real-time display of the user's ECG signals. For example, the processing device 311 may include firmware/logic for processing and displaying the signals received from the electrodes 314 (or the touch pads 318 and 319) in a similar manner as the processing device of computing device 320. In this embodiment, all connections may be hard wired or wireless. The memory 312 of monitoring device 310 may include instructions for causing the processing device 311 to process the ECG signals from a user contacting the electrodes 314 and displaying the heart-signals on a display (not shown) located on an exterior surface of the monitoring device 310. The transceiver 313 may be used to transmit the processed signal to the computing device 320 and/or another computing device where a medical professional may also view the recording. Alternatively, once in receipt of the data, the computing device 320 may send the data to a medical professional using well known communications and data transfer technologies.

Upon the computing device 320 receiving the ECG signals measured by the electrodes 314 and deriving/computing any number of additional leads as discussed herein, the computing device 320 may display the generated leads to the user on a display of the computing device 320.

The computing device 320 may also include a database 323 which may be used to store ECG data of the user (as well as other users). The ECG data of the user may include ECG signals measured during various ECGs performed over time. The computing device 320 may make the user's ECG data available (e.g., at a net storage location) via a URL. The URL may be encoded into the bar code 320 so that when the user scans the bar code 320 using e.g., their smart phone or other appropriate computing device, they may be redirected (e.g., via a browser or other similar software running on the computing device) to the URL where their ECG data is available.

FIG. 4F is a flow diagram of a method 400 of obtaining and providing remote ECG data using an ECG device with a portable form factor, in accordance with some embodiments. Method 400 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof in some embodiments, the method 400 may be performed by a computing device (e.g., monitoring device 310 illustrated in FIGS. 4A to 4D).

Method 400 begins at block 405, wherein processing logic detects an electrical signal from each of a set of electrodes disposed on a front and rear surface of a layered housing having a portable form factor. In some examples, the form factor may be approximately the size of a credit card (e.g., for ease of portability). In some embodiments, the housing may have a level of bending stiffness or flexibility that permits a user to place it in a purse or wallet in a similar manner to how a normal credit or debit card is stored and carried. The ECG monitoring device can be used by a user/patient, and provides ECG data to a user on a near instantaneous basis. In some examples, the electrodes may be located on the housing to allow a right hand to touch a first electrode, a left hand to touch second electrode, and a third part of the body (e.g., a leg, chest, etc.) to touch a third electrode (e.g., on a backside of the housing). In some examples, the electrodes are disposed in various layers of the housing. For example, the first and second electrodes may be disposed and exposed within the first layer while the third electrode may be disposed and exposed within a second layer of the housing.

At block 410, the processing logic measures the electrical signals from each of the set of electrodes to record three ECG leads. For example, the processing logic may perform analog to digital conversion of the electrical signals as well as any additional signal processing/cleaning functions necessary before transmitting the measured/digitized/processed electrical signals to a database or other computing device. At block 415, the processing logic calculates additional ECG leads using the recorded three ECG leads. For example, the ECG monitoring device may acquire leads I, II, and III and derive leads aVR, aVL, and aVF, as described in detail above. However, any other combination of leads is possible. The ECG monitoring device may subsequently generate a 12-lead ECG using the three measured leads.

At block 420, the processing logic provides a user with access to the data corresponding to the three ECG leads and the additional ECG leads via scanning of a bar code disposed on a surface of the layered housing. For example, the computing device of the ECG may be coupled to a database to store ECG data of the user (as well as other users). The ECG data of the user may include ECG signals measured during various ECGs performed over time. The computing device 320 may make the user's ECG data available (e.g., at a net storage location) via a URL. In some examples. the URL may be encoded into a bar code, or other scannable indicator, so that when the user scans the bar code, e.g., using their smart phone or other appropriate computing device, they may be redirected (e.g., via a browser or other similar software running on the computing device) to the URL where their ECG data is available.

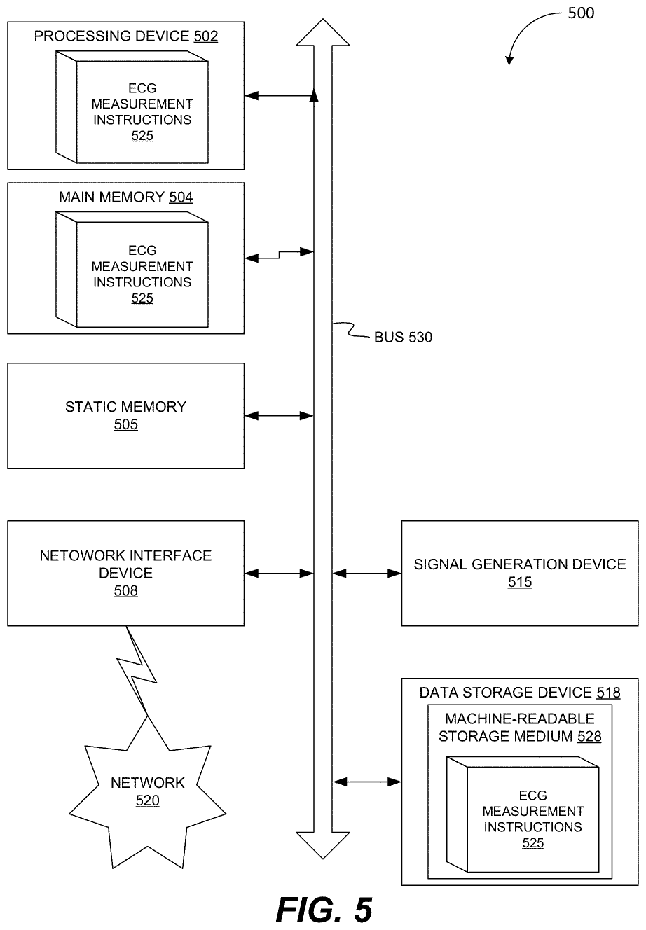

FIG. 5 illustrates a diagrammatic representation of a machine in the example form of a computer system 500 within which are stored a set of instructions for causing the monitoring device 310 to perform any one or more of the methodologies discussed herein for taking a 6-lead ECG of a user.

In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 500 may be representative of a server.

The exemplary computer system 500 includes a processing device 502, a main memory 504 (e.g., read-only memory (ROM), flash memory, dynamic random access memory (DRAM), a static memory 506 (e.g., flash memory, static random access memory (SRAM), etc.), and a data storage device 518, which communicate with each other via a bus 530. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Computing device 500 may further include a network interface device 508 which may communicate with a network 520. The computing device 500 also may include a video display unit 510 (e.g., a liquid crystal display (LCD) or a cathode ray tube (CRT)), an alphanumeric input device 512 (e.g., a keyboard), a cursor control device 514 (e.g., a mouse) and a signal generation device 515 (e.g., a speaker). In one embodiment, video display unit 510, alphanumeric input device 512, and cursor control device 514 may be combined into a single component or device (e.g., an LCD touch screen).

Processing device 502 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 502 may also be one or more special-purpose processing devices such as an application specific integrated circuit (ASIC), a field programmable gate array (FPGA), a digital signal processor (DSP), network processor, or the like. The processing device 502 is configured to execute 6-lead ECG measurement instructions 525, for performing the operations and steps discussed herein.

The data storage device 518 may include a machine-readable storage medium 528, on which is stored one or more sets of 6-lead ECG measurement instructions 525 (e.g., software) embodying any one or more of the methodologies of functions described herein. The 6-lead ECG measurement instructions 525 may also reside, completely or at least partially, within the main memory 504 or within the processing device 502 during execution thereof by the computer system 500; the main memory 504 and the processing device 502 also constituting machine-readable storage media. The 6-lead ECG instructions 525 may further be transmitted or received over a network 520 via the network interface device 508.

While the machine-readable storage medium 525 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

What is claimed is:

1. An apparatus comprising:
a housing having a credit card form factor;
a set of electrodes to generate signals corresponding to electrical activity of a heart of a user;
a memory; and
a processing device operatively coupled to the memory, the processing device to:
perform a multi-lead electrocardiogram (ECG) using the set of electrodes; and
transmit data corresponding to the multi-lead ECG to a computing device.

2. The apparatus of claim 1, wherein to perform the multi-lead ECG, the processing device is to:
in response to the user making contact with each of the set of electrodes, measure a signal generated by each of the set of electrodes to record three ECG leads; and
synthesize, using an algorithm, one or more additional ECG leads using the recorded three ECG leads, wherein the data corresponding to the multi-lead ECG comprises the recorded three ECG leads and the one or more additional ECG leads.

3. The apparatus of claim 1, wherein the housing has a multi-layered structure comprising:
a lower layer having the set of electrodes, the memory, and the processing device mounted thereon; and
an upper layer to expose one or more of the set of electrodes.

4. The apparatus of claim 3, wherein a first electrode of the set of electrodes is mounted on an underside of the lower layer.

5. The apparatus of claim 4, wherein the first electrode protrudes beyond an edge of the underside of the lower layer.

6. The apparatus of claim 3, wherein a first electrode and a second electrode of the set of electrodes is mounted on a topside of the lower layer and are exposed by the upper layer.

7. The apparatus of claim 1, further comprising:
a bar code, to provide access to the data corresponding to the multi-lead ECG in response to being scanned by any appropriate device.

8. The apparatus of claim 1, wherein the housing comprises:
a first layer;
a second layer; and
an inlay sandwiched between the first and second layers, wherein the set of electrodes, the memory, and the processing device are integrated with the inlay.

9. The apparatus of claim 1, wherein the housing comprises a polymer material.

10. The apparatus of claim 1, wherein the housing comprises a plastic material.

11. A system comprising:
a monitoring device comprising:
a housing having a credit card form factor;
a set of electrodes to generate signals corresponding to electrical activity of a heart of a user;
a memory; and
a processing device operatively coupled to the memory, the processing device to:
perform a multi-lead electrocardiogram (ECG) using the set of electrodes; and
transmit data corresponding to the multi-lead ECG; and
a computing device to:
receive the data corresponding to the multi-lead ECG;
process the data corresponding to each lead of the multi-lead ECG to generate a set of additional ECG leads; and
display each lead of the multi-lead ECG and the set of additional ECG leads.

12. The system of claim 11, wherein to perform the multi-lead ECG, the processing device is to:
in response to the user making contact with each of the set of electrodes, measure a signal generated by each of the set of electrodes to record three ECG leads; and
synthesize, using an algorithm, one or more additional ECG leads using the recorded three ECG leads, wherein the data corresponding to the multi-lead ECG comprises the recorded three ECG leads and the one or more additional ECG leads.

13. The system of claim 11, wherein the housing has a multi-layered structure comprising:
a lower layer having the set of electrodes, the memory, and the processing device mounted thereon; and
an upper layer to expose one or more of the set of electrodes.

14. The system of claim 13, wherein a first electrode of the set of electrodes is mounted on an underside of the lower layer.

15. The system of claim 14, wherein the first electrode protrudes beyond an edge of the underside of the lower layer.

16. The system of claim 13, wherein a first electrode and a second electrode of the set of electrodes is mounted on a topside of the lower layer and are exposed by the upper layer.

17. The system of claim 11, further comprising:
a bar code, to provide access to the data corresponding to the multi-lead ECG in response to being scanned by any appropriate device.

18. The system of claim 11, wherein the housing comprises:
a first layer;
a second layer; and
an inlay sandwiched between the first and second layers, wherein the set of electrodes, the memory, and the processing device are integrated with the inlay.

19. The system of claim 11, wherein the housing comprises a polymer material.

20. The system of claim 11, wherein the housing comprises a plastic material.
